(19) United States
(12) Patent Application Publication
(10) Pub. No.: US 2025/0041861 A1
(43) Pub. Date: Feb. 6, 2025

(54) BIOMEDICAL MEASURING DEVICES, SYSTEMS, AND METHODS FOR MEASURING ANALYTE CONCENTRATION

(71) Applicant: Jana Care, Inc., Watertown, MA (US)

(72) Inventors: Michal Depa, Beaconsfield (CA); Ashok a Kumar, Medford, MA (US); Sidhant Jena, Delhi (IN); K.C. Chen, Anaheim, CA (US); Yunyuan Vivian Wang, Anaheim, CA (US); Stephen L. Chen, Anaheim, CA (US)

(73) Assignees: Jana Care, Inc., Watertown, MA (US); Teco Diagnostics, Anaheim, CA (US)

(21) Appl. No.: 18/921,238

(22) Filed: Oct. 21, 2024

Related U.S. Application Data
(63) Continuation of application No. 15/883,410, filed on Jan. 30, 2018, now abandoned.

(51) Int. Cl. B01L 3/00; G01N 21/78; G01N 33/72

(52) U.S. Cl. CPC B01L 3/502761; B01L 3/5023; G01N 21/78; G01N 33/723; B01L 2300/0681; B01L 2300/069; B01L 2300/0825

(57) ABSTRACT

A biomedical measuring device, such as a test strip, has a simple structure, by which analyte can be measured easily using a small amount of specimen. In embodiments, the test strip generally includes a plastic first film layer having structure defining an aperture for retaining the reacting components, a porous membrane coupled to an inner-facing surface of the first film layer and configured to reduce background signal, an absorbent pad coupled to the porous membrane and first film layer to sandwich the porous membrane therebetween, the absorbent pad being configured for rapid absorption, and a plastic second film layer coupled to the absorbent pad, the second film layer being configured to provide a barrier to prevent liquid from leaking out of the test strip during use. The test strip can be easily used with an optical sensing device coupled to or containing an analyzer device (or reader device) for quickly detecting and measuring the analyte concentration. In a particular embodiment, the analyte comprises glycated hemoglobin or HbA1c, and the optical reader device is configured to determine HbA1c concentration.

### RELATED APPLICATION

This application is a continuation of U.S. application Ser. No. 15/883,410 filed Jan. 30, 2018, which is hereby fully incorporated herein by reference.

### FIELD OF TECHNOLOGY

Embodiments related generally to monitoring diabetes, and more specifically to measuring a level of glycated hemoglobin in a blood sample using a test strip and a portable optical sensing device paired to a mobile device.

### BACKGROUND

Diabetes mellitus is a chronic disease caused by dysfunction of insulin regulation, resulting in elevated blood glucose levels and its associated complications such as diabetic retinopathy, renal failure, foot ulceration, and heart disease. Two commonly tested markers for monitoring diabetes are glucose and glycated hemoglobin (HbA1c). Long-term glucose assessment using HbA1c biomarker is advantageous because it eliminates the large fluctuations that occur daily in the blood glucose concentrations. The American Diabetes Association recently has set a ratio of HbA1c over total hemoglobin of greater than 6.5% as an indication of diabetes, while HbA1c levels of 5.7-6.4% are an indication of an increased risk for diabetes.

A variety of methods have been proposed for measuring HbA1c concentration in blood. They can be broadly divided into four categories: 1) ion-exchange chromatography; 2) immunoassay; 3) boronate affinity; and 4) enzymatic methods. To render the HbA1c test more affordable and easy to be used by medical professionals, a point-of-care (POC) HbA1c test using a simple assay and device is desired.

Among the current POC HbA1c devices, the boronate affinity method is most commonly used. The boronate affinity separation of glycated hemoglobin from a blood sample was developed in the 1980s based on the ability of boronic acids (usually derivatives of phenylboronic acid) to form cyclic esters with 1, 2-cist-diols presented in the glucose chain of HbA1c molecule (see FIG. 1). The separation of HbA1c and HbA₀ can be done by attaching the boronic acid to a solid support or carrier, such as, for example, beads, acrylic particles, magnetic particles, membranes, or the like, followed by a simple washing or filtration procedure, as shown in FIG. 1.

U.S. Pat. Nos. 5,506,144 5,702,952, 5,631,364, and 5,919,708, demonstrate that HbA1c, when conjugated with a blue dye containing boronic acid, can be distinguished from the total hemoglobin based on the color differences. The Afinion and Nycocard devices, originally available from Axis-Shield Diagnostics, and now available from Alere, which has been recently acquired by Abbott, are commercial POC devices based on this technology which include an analyzer and test cartridges. The blood sample is inserted and mixed with solution pre-packed in the cartridge by the machine. The reaction mixture is soaked through a filter membrane and all precipitated hemoglobin including dye conjugate-bound HbA1c and unbound Hb get stopped by the membrane. The cartridge also contains a wash buffer chamber to remove excess dye conjugate from the membrane. The analyzer then measure the reflectance of the blue (i.e. glycated hemoglobin) and the red (i.e. total hemoglobin) color intensities on the membrane and calculates the fraction of HbA1c in the sample

U.S. Patent Application Publication No. 2009/0093012 is directed to the commercially-available Clover A1c test cartridge, available from Infopia Co., Ltd. of South Korea. The test cartridge is composed of a sample colleting leg and a reagent pack pre-filled with reaction solution and washing solution. The reaction solution contains agents that lyse red blood cells and bind hemoglobin specifically, as well as a boronate resin that binds glycated hemoglobin. When the cartridge is inserted in the machine, it is rotated by the machine, which mixes the blood sample collected in the sample collecting leg with reaction solution. The total hemoglobin is measured by an optical sensor. The subsequent rotation allows wash buffer to remove unbound conjugate and bound HbA1c conjugate is measured by the optical sensor. The analyzer then calculates the fraction of HbA1c. Although sophisticated, the cost of this kind of cartridge and corresponding machine are prohibitively high.

An attempt to reduce a production cost of the HbA1c test is described in U.S. Pat. No. 8,172,994, assigned to Ceragem. U.S. Pat. No. 8,172,994 discloses the process of forming a plurality of reaction elements on a first substrate in which forming a reaction element includes forming at least two first electrodes on a first side of the first substrate, forming a second electrode on a second side of the first substrate, in which the second electrode transmits an electrical signal to a measuring device, forming a via hole through the first substrate for electrically connecting the first electrodes on the first side of the first substrate to the second electrode on the second side of the first substrate, and applying an assay reagent to the first electrodes on the first side of the first substrate. The first substrate is then cut into a plurality of reaction elements. At least one cavity is formed, each with space for a capillary, on one side of a second substrate, and at least one capillary is formed by attaching the first side of at least one reaction element into at least one of the cavities in the second substrate.

However, the stacked material in U.S. Pat. No. 8,172,994 requires cutting the material into individual units and placed in a housing disk one by one and mounted. This is a very labor consuming manual manufacturing process or requires high cost automatic machine to perform the assembly. Moreover, the mounting process is very crucial for the quality of the device. If the plastic housing disk does not compress the stacked material tight enough, the loaded sample will leak through the edge of the hole on the plastic disk and compromise the accuracy of the assay.

Porous membranes have been used in biomedical devices for detecting the analyte by either separating the analyte from the matrix or specifically binding the analyte. For example, PCT Application Publication No. WO 2002-090995A2 discloses a membrane filter cartridge which separates serum from blood cells and separates precipitant from suspension. PCT Application Publication No. WO 1990-002950A1, and U.S. Patent Application Publication No. 2012/0302456 disclose membrane filter-based enzyme linked immunosorbent assays (ELISA) plates coated with antibody, and which bind specifically the analyte and separate microspheres from washing buffer. However, these technologies are high cost and have complicated manufacturing processes. Also, these types of devices require a larger volume of reagent and specimen, and a larger optical detection unit.

There remains a need for a cost effective, simplified measuring device or test strip for measuring analyte concentration.

### SUMMARY OF THE INVENTION

Embodiments comprise a biomedical measuring device, such as a test strip, having a simple structure, by which analyte can be measured easily using a small amount of specimen. The test strip uses minimized material cost and does not require complicated automation for production such that the test strip is cost efficient. The test strip can be easily used with an optical sensing device coupled to or containing an analyzer device (or reader device) for quickly detecting and measuring the analyte concentration. In a particular embodiment, the analyte comprises glycated hemoglobin or HbA1c, and the optical reader device is configured to determine HbA1c concentration.

In embodiments, the test strip generally includes a plastic first film layer having structure defining an aperture for retaining the reacting components, a porous membrane coupled to an inner-facing surface of the first film layer and configured to reduce background signal, an absorbent pad coupled to the porous membrane and first film layer to sandwich the porous membrane therebetween, the absorbent pad being configured for rapid absorption, and a plastic second film layer coupled to the absorbent pad, the second film layer being configured to provide a barrier to prevent liquid from leaking out of the test strip during use.

The layers can be bonded together by any of a variety of bonding techniques, such as, for example, adhesives, heat sealable materials, or ultrasonic welding. In a particular embodiment, an adhesive layer is present between the first film layer and the porous membrane, and an additional adhesive layer is present between the porous absorbent pad and the second film layer.

In an embodiment, the first and second film layers define the two outermost layers of the composite test strip, however, in alternative embodiments, additional layers and/or coatings can be incorporated as desired.

A kit and a method for using the kit for monitoring diabetes, according to embodiments of the invention, includes a plurality of test strips, a plurality of reagent vials, the reagent being configured to precipitate glycated hemoglobin and total hemoglobin from a blood sample and to bind glycated hemoglobin to a dye, a plurality of washing solutions to remove unconjugated dye from the test strip during testing, and a set of instructions for preparing the test strip for measurement using an optical sensing device coupled to or incorporated into an analyzer device.

According to embodiments, a method for monitoring diabetes can include obtaining a blood sample, reacting the blood sample with a reagent configured to precipitate glycated hemoglobin and total hemoglobin from a blood sample and to bind glycated hemoglobin to a dye, applying the reacted blood sample to a test strip, washing the test strip with a washing solution to remove unconjugated dye, and inserting the reacted test strip into an optical sensing device coupled to or incorporated into an analyzer device for measurement and analysis.

In a particular embodiment, the method further includes installing an application on a mobile device, pairing the mobile device with the optical sensing device, and collecting, reading, and/or analyzing the data in the application on the mobile device.

The devices, systems, and methods according to embodiments provide a quick, portable, minimally invasive, and cost efficient mechanism for measuring an analyte for monitoring diabetes in a patient compared to those of the prior art.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a mode of conjugation between phenylboronic acid and protein HbA1c, according to the prior art.

FIG. 2 is an exploded view of a test strip assembly according to an embodiment of the invention.

FIG. 3A is a bottom view of the test strip assembly of FIG. 2.

FIG. 3B is a top view of the test strip assembly of FIG. 2.

FIGS. 4(a)-(d) are cross-sectional views of a first film layer according to an embodiment of the present invention

FIG. 5 is a top view of an optical or color sensing device coupled to an optical reader or analyzer device, the sensing device including the test strip assembly of FIG. 2 inserted therein.

FIG. 6 depicts a kit for monitoring diabetes using the test strip assembly of FIG. 2, according to an embodiment of the invention.

FIG. 7 is a flow chart of a method of monitoring diabetes according to an embodiment of the invention.

FIG. 8 is a graph correlating glycated hemoglobin concentrations measured using an embodiment of the invention for an HbA1c device and a Bio-Rad Variant II Turbo™ device.

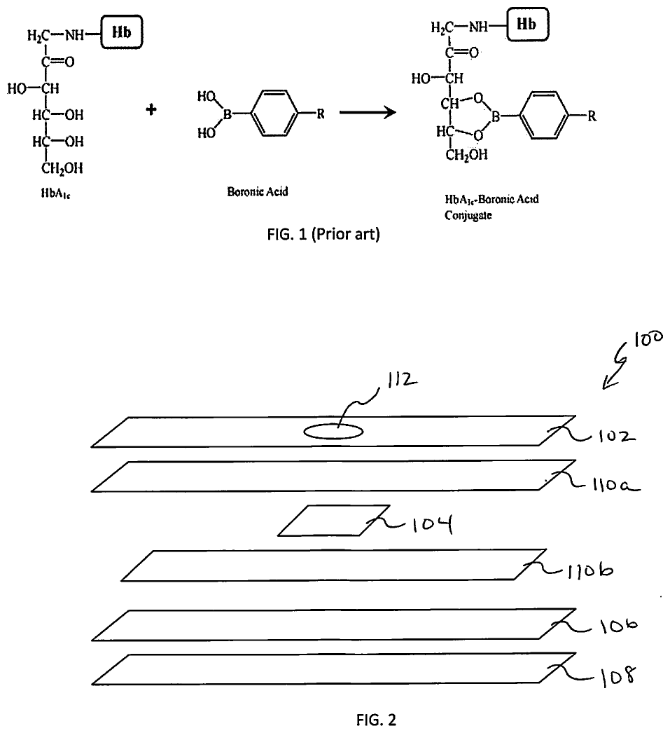

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The embodiments described below are not intended to be exhaustive or to limit the invention to the precise forms disclosed in the following detailed description. Rather the embodiments are chosen and described so that others skilled in the art may appreciate and understand the entire disclosure.

Referring to FIG. 2, a biomedical measuring device comprises a composite test strip assembly 100 used for applying a sample and for inserting such sample laden strip into an optical sensing and reading apparatus for analysis of the sample. In the embodiment depicted in FIG. 2, test strip assembly 100 comprises six layers. In alternative embodiments, more or less than six layers can be contemplated.

Test strip assembly 100 can comprise a sample receiving and detection first or top film layer 102, a porous membrane 104 coupled to top film layer 102, an absorbent pad 106 coupled to top film layer 102 and porous membrane 104, and a second or bottom film layer 108. Adhesive layer 110a is included to bond top film layer 102 to porous membrane 104 and absorbent pad 106, and adhesive layer 110b is included to bond bottom film layer 108 to absorbent pad 106.

Top film layer 102 can be formed from a plastic or polymeric material that exhibits a balance between a moderate flexural modulus (e.g. from about 100,000 to about 600,000 psi), and good tensile strength (e.g. from about 3000 to about 15000 psi). This allows for ease in manufacturing, yet is still rigid enough for performing the assay. Suitable materials include, for example, acetal copolymer, acrylic, nylon, polyester, polypropylene, polyphenylene sulfide, polyetheretherketone, poly(vinyl chloride), or combinations thereof.

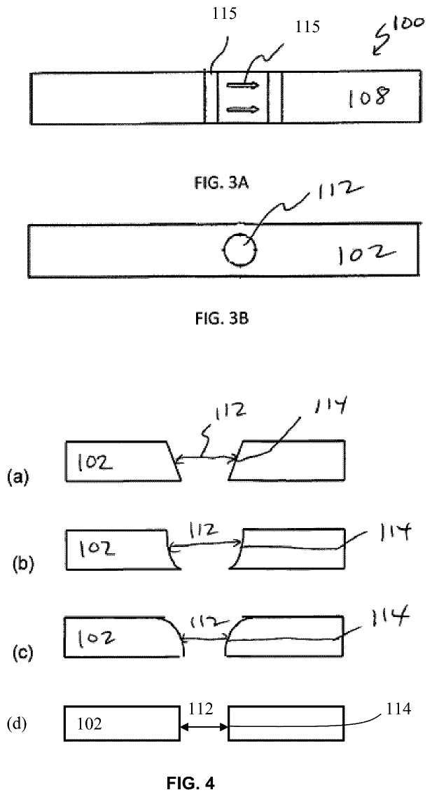

In embodiments, and referring to FIGS. 3A and 3B, a top film layer 102 is rectangular and shape, and has a length in ranging from about 30 mm to about 80 mm so that it retains its rigidness. A thickness of top film layer 102 can range from about 0.1 mm to about 1 mm, so that when an aperture 112 is present in top film layer 102, a reservoir is created for the application of a sample mix. More particularly, aperture 112 is formed into layer 102 by any of a variety of standard cutting techniques, such as, for example, die cutting or punching, laser cutting, or the like. Aperture 112 can be circular, as depicted, having a diameter ranging from about 2 to about 6 mm, allowing the reservoir to hold up a sample volume in a range from about 10 to about 50 ul. One of ordinary skill in the art would recognize that other aperture geometries can also be contemplated, including, for example, oval, square, rectangular, triangular, etc., with dimensions such that a similar sample volume can be contained.

When aperture 112 is formed, certain structure of the sidewall is desired for fast and smooth sample flow. Referring to FIGS. 4(a)-4(c), a sectional view of various sidewall geometries of aperture 112 is illustrated. For example, a sidewall 114 of aperture 112 can be tapered as shown in FIG. 4(a), concave as shown in FIG. 4(b), convex as shown in FIG. 4(c), or substantially vertical as shown in FIG. 4(d).

Referring back to FIG. 2, porous membrane 104 is made of a selectively porous material. In embodiments, porous membrane 104 can retain bound hemoglobin and/or glycated hemoglobin particles, while allowing the unbound dye to penetrate through. Porous membrane 104 can comprise nitrocellulose, cellulose acetate, polyethylene, polyester, polyether sulfone (PES), and/or polycarbonate. A desired pore size comprises a range of from about 0.2 to about 20 μm.

Since membrane 104 is porous, when it is wetted with biomedical reagent, it becomes semi-transparent. Therefore, any layer underneath membrane 104, such as absorbent pad 106, is colored, it could potentially interfere with the optical apparatus reading of membrane 104 during analysis with an optical measuring apparatus. Therefore, membrane 104 can optionally be impregnated with a filler or whitening agent, such as titanium dioxide, to provide opacity to membrane 104 to reduce background signal for a better reflectance signal and test accuracy for the optical measuring apparatus.

Absorbent pad 106 provides capillary force for directing flow of the sample mix toward the top and the bottom of composite strip assembly 100 while the sample mix is penetrating through membrane 104. Absorbent pad 106 can comprise one-direction or multi-direction woven fiber, or alternatively a non-woven material such as a spun-bonded or plexifilamentary absorbent material. The fiber material can comprise, for example, nylon, fiberglass, a superabsorbent polymer such as a hydrogel, cellulose, or combinations thereof. In particular embodiments, a desired thickness of pad 106 is in a range of from about 0.1 to about 1 mm, and a length of pad 104 can be about 10 to about 45 mm shorter than the length of adhesive layers 110a and 110b to enable the adhesives to bind both sides of the absorbent pad 106 and hold the strip together.

Bottom film layer 108 comprises a support layer formed of a polymeric or plastic film material that is not transparent. For example, bottom film layer can comprise polyethylene, polyvinyl chloride (PVC), polypropylene, polyethylene terephthalate (PET), polytetrafluoroethylene (PTFE), or combinations thereof. In particular embodiments, a desired thickness of film layer 108 is in a range of from about 0.1 to about 10 mm.

Adhesive or bonding layers 110a, 110b can be comprises of the same or different materials, and can comprise a continuous layer or a spot-coated layer. As mentioned about, layer 110a provides binding between layers 102, 104 and 106, while layer 110b provides binding between layers 106 and 108.

In an embodiment, layers 110a, b can comprise a thin film or coating of acrylic polymer. In other embodiments, layers 110a, 110b can comprise a coating or film of a polymeric heat sealable material such as polyethylene, or can comprise any of a variety of curable adhesive such as, for example, radiation-curable adhesives, heat-cured adhesives, moisture-cured adhesives, epoxies, or any combination thereof. In a particular embodiment, a desired thickness of layers 110a, 110b, when comprising a film, is in a range of from about 0.01 to about 0.5 mm.

In an embodiment, the first and second substrates define the two outermost layers of the composite device, however, in alternative embodiments, additional layers and/or coatings can be incorporated as desired. In one particular embodiment, and referring to FIG. 3A, bottom film layer 108 (and/or optionally top film layer 102) comprises printed indicia 115 thereon. Printed indicia 115 can comprise any of a variety of text and/or graphics such as, for example, brand names, logos, instructions, readout messages, warnings, or any combination thereof. In an embodiment, printed indicia 115 can comprise, for example, text and/or graphics, such as arrows, indicating how test strip assembly 100 is to be inserted into an optical sensing device for measurement.

In embodiments, test strip assembly 100 can be manufactured individually as discrete test strips. Alternatively, a plurality of test strip assemblies can be manufactured in roll form or in a large card format, and upon assembly, individual test strip assemblies are converted or cut therefrom.

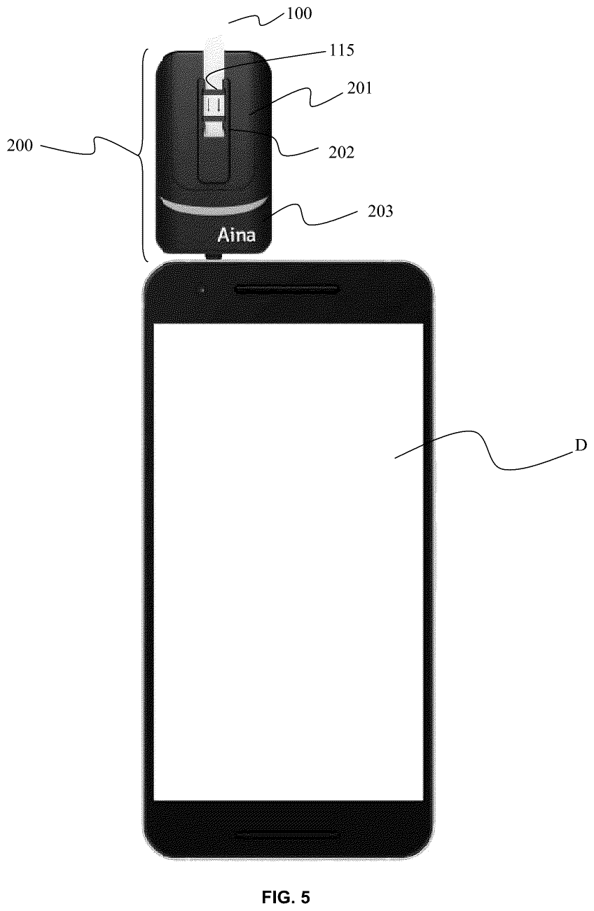

Referring now to FIG. 5, test strip assembly 100 is read using an optical or color sensing device 200 configured to be coupled to a measuring or analyzing instrument D for analyzing a specimen, such as a blood sample.

In a particular embodiment, sensing device 200 can comprise a hand-held reflectance based-optical sensor device, such as a colorimeteric sensor device. Once such suitable sensing device is commercially available as the Aina Device, available from the applicant of the present disclosure, and which is described in U.S. application Ser. No. 14/997,749 entitled “Mobile Device Based Multi-Analyze Testing Analyzer for Use in Medical Diagnostic Monitoring and Screening,” incorporated herein by reference in its entirety. In embodiments, sensing device 200 connects to any of a variety of mobile devices, such as smart phones or tablets, through the audio jack or jack plug of the mobile device. Although generally referred to herein as “jack plug” for sake of convenience, a jack plug can include any wired or wireless communication element including, but not limited to, universal serial bus (USB), including micro USB and mini USB, Bluetooth®, near field communication (NFC), or WLAN (any IEEE 802.11 variant). The mobile device includes an application that runs on the mobile device for analyzing data generated by device 200.

Device 200 generally includes an adapter 201 coupled to an optical sensing body 203 containing optical or color sensing components within (internal, not shown, and as described, for example, in U.S. application Ser. No. 14/997,749). Adapter 201 enables the test strip assembly 100 to align with the optical sensing components housed within optical sensing body 203. Adapter 201 includes structure defining a test strip insertion area 202, such as a slot or channel, for inserting test strips, such as test strip assembly 100 described in the previous section. When inserted, test strip assembly 100 is illuminated by one or several light sources (internal, not shown) housed within body 203. The light reflects from membrane layer 104 of the test strip containing the analyte, which is then measured by a light sensor, such as a photodiode contained in body 203 of device 200. The reflected color value is then relayed to the mobile device D where it is processed and analyzed by software algorithms contained in the application installed on the mobile device to produce an HbA1c reading. At each step, appropriate instructions are displayed on the mobile device's screen to guide the user in performing the test, which will be discussed in more detail infra.

In an embodiment, sensing device 200 includes illumination light sources (internal, not shown) that allow for bright and consistent illumination, as described in U.S. patent application Ser. No. 14/997,749, incorporated by reference above. One such suitable source of illumination includes through-hole LEDs, which are cost-effective if high luminosity levels are required. To effectively measure the HbA1c reaction on test strip assembly 100 described in the previous section, sensing device 200 can comprise at least two separate illumination light sources at different wavelengths. For example, a first illumination source has a dominant wavelength between 600 nm and 650 nm, and a second illumination source has a dominant wavelength between 450 nm and 490 nm. These are required to read the level of glycated hemoglobin via the dye that is bound to membrane layer 104 as described in the previous section, and the level of total hemoglobin contained in the sample, depending on the testing method. The HbA1c reading can then be determined by taking a ratio of the glycated hemoglobin level to the total hemoglobin level, as will be discussed in more detail infra.

As described above, test strip assembly 100 can contain printed indicia 115 that aligns with features on test strip insertion area 202 of sensing device 200, which allows a user to visually confirm that strip assembly 100 is inserted properly by virtue of the features being aligned, as depicted in FIG. 5.

Optionally, in an embodiment, as sensing device 200 senses and transmits reflected color data to the mobile device for processing and analysis, the software on the mobile device performs various boundary checking to ensure that strip assembly 100 is inserted properly at the different steps, and is not moved during the analysis. These algorithms may include, for example, simple checks such as checking if the reflected value is within a certain expected range, which can be performed simultaneously for the different wavelengths in which test strip assembly 100 is being analyzed.

In another embodiment, movement of test strip assembly 100 during analysis can be assessed by first measuring the reflected color value of test strip assembly 100 for a pre-determined amount of time, computing an averaging statistic such as an average or a median on this data, and then comparing subsequent reflected color values received by the software running on the mobile device against the previously computed statistic. If the subsequent reflected color values received are not within a pre-determined range from the averaging statistic, an error is shown to the user on the screen of the mobile device, sounded by the mobile device, or shown or sounded by optical reader 200, indicating to the user that the test strip was moved or otherwise disturbed during the test.

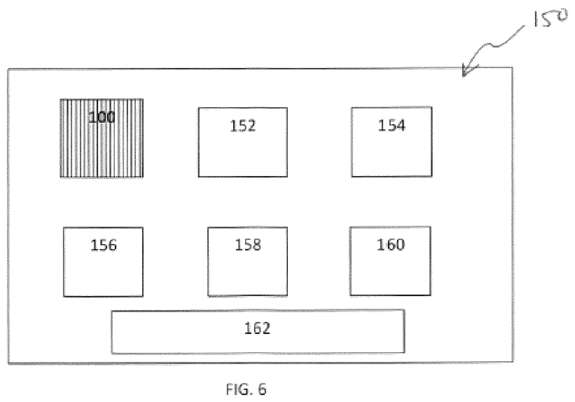

Now referring to FIG. 6, in an embodiment, a monitoring test kit 150 for monitoring diabetes comprises a plurality of test strip assemblies 100 described above, a plurality of reagent vials 152 for conjugating hemoglobin, glycated hemoglobin, or both, a plurality of wash buffer vials 154 for removing any unbound reagent, a plurality of sample collection vials 156, such as capillary blood tubes, a plurality of pipette tips 158, a plurality of lancets 160 for obtaining the sample from a user, and/or instructions for use 162. One of ordinary skill in the art would recognize that the various components can be packaged in a single packaging container such as a box, or multiple containers or boxes as desired. For example, in a non-limiting embodiment, a first package can include components that can be stored at room temperature, and a second package can include components that are preferably stored or required to be stored at temperatures less than room temperature, such as cooled, refrigerated, and/or freezing environment. In another embodiment, certain components, such as lancets, can be supplied separately, and not as part of kit. It is appreciated that any combination of packaging configurations as desired or required can be contemplated.

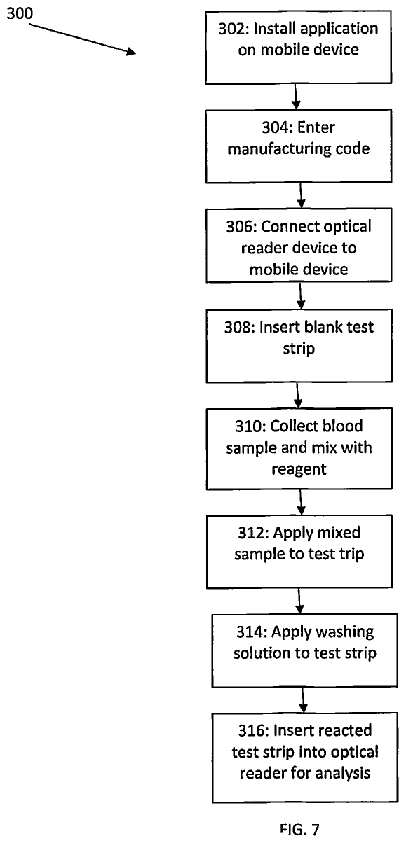

During the first step of the test, the user can be instructed via instructions 162 to enter in a user interface on the mobile device a code number corresponding to the manufacturing lot of the HbA1c reagent kit 150. The software running on the mobile device then can either download a configuration file from a remote server via the internet or load it if it is already available locally on the mobile device storage. This configuration file can contain various parameters, such as the illumination light sources to use in the analysis, their brightness and sampling frequency, the duration of the analysis, the statistic used to summarize the data collected over the duration of the analysis as well as the calibration curve that maps these summary statistics to the equivalent HbA1c readings. The summary statistic can be a median, average, maximum, minimum, or other statistic that summarizes data collected over a duration of time into a single value. An advantage of collecting data over a certain time duration and computing a summarizing statistic on it is to remove any variations caused by noise emanating from the system or caused by slight variations of the reacted color of the HbA1c test strip assembly 100 once the sample is applied to it. During the analysis, this summarizing can be done separately for data collected using different wavelengths (corresponding to different illumination light sources). In one embodiment, measurements of the HbA1c test strip assembly 100 are performed in two different wavelengths, then the reflected color data measured at each wavelength is summarized using of the statistics described above, before being combined into a single value by taking their ratio or another similar method. This final value can be used as the input to a calibration curve depicted in FIG. 7 to obtain an HbA1c reading that is displayed to the user on the mobile device screen.

Since there is a separate configuration file for each manufacturing lot of HbA1c reagent kits 150, a customized set of analysis parameters, as described above, can be established for each such manufacturing lot. This includes the brightness of the light sources, the sampling frequency, the duration of the analysis and the statistics used to summarize the data before applying the calibration curve to obtain a final HbA1c reading. In addition, since the calibration curves can be downloaded from a remote server, these can be updated over time to optimize performance of deployment systems in the field, for instance by taking into account natural aging of the reagent kits 150 over time.

In another embodiment, the manufacturing lot specific configuration file can also contain nominal values for the reflected color values of the blank test strips. This would remove the need from having to measure a blank test strip at the first step of the test with the assumption that the manufacturing variability between the test strips is small enough to allow for this. Instead of measuring the blank test strip, the nominal values could be loaded from the configuration file at the beginning of the test and used instead.

The optics of each sensing device 200 can vary slightly because of the individual characteristics of different components, such as the illumination sources, the light sensor and the overall geometry of the optical system. In order to compensate for such variations between readers, test strips with a constant color, called mock strips, can be measured on each sensing device 200 in order to characterize each reader's optics. In an embodiment, at least two mock strips that emulate the colors of a blank and reacted HbA1c test strip, respectively, can be used, as to effectively characterize the optical system of each sensing device 200 across the relevant reflected color measuring range. These measured reflected color values can be stored in the non-volatile memory of each sensing device 200, so they can later be sent to the software on the mobile device that connects to sensing device 200, and used to algorithmically compensate the reflected color values subsequently measured by each sensing device 200 to each other. This can effectively compensate away the differences in optics in the different sensing devices 200 in the software, allowing the same calibration curve to be used by all devices 200.

In one particular embodiment, test strip assembly 100 and kit 150 are configured to utilize the boronate affinity method. In this embodiment, reagent vials 152 contain a lysing agent and a blue boronic acid conjugate. When blood is collected via lancets 160 and collection vials 156 and added to the reagent, erythrocytes are lysed and hemoglobin precipitates. The boronic acid conjugates binds to the glycosylated hemoglobin. An aliquot of the reaction mixture is applied, via pipette tips 158, to the test strip and all the precipitated hemoglobin, conjugate-bound and unbound, remains on top of the porous membrane of test strip assembly 104. Any unbound boronate is removed with the wash buffer from wash buffer vials 154. The precipitate (or analyte) is evaluated by measuring the blue (glycosylated hemoglobin) and the red (total hemoglobin) color intensity using two wavelengths with the optical sensing device described previously. The ratio between the blue and red color intensities is proportional to the percentage of glycosylated hemoglobin in the sample.

Referring now to FIG. 7, to perform a method 300 of analyzing a concentration of an analyte, and more specifically for determining HbA1c levels of a patient, at 302, a user opens a test application install on a mobile device, such as a smart phone or table, and as described above. At 304, the user is asked by the application to enter or scan a manufacturing code printed on a reagent bag containing reagent vials or elsewhere in kit 150 being used, which allow the application to load pre-determined lot-specific calibration curves.

At 306, the user connects an optical or color sensing device, such as device 200 described above, to the mobile device, and at 308, inserts a blank test strip, such as strip assembly 100 described above, into the sensing device to obtain an initial reading or blank signal that is transmitted to the application running on the mobile device.

At 310, the user collects and adds a volume of blood, such as, for example, from about 1 to about 10 μL, or a volume as specified by instructions 162, of venous or capillary whole blood from the patient to a reagent vial, which is then mixed for a desired amount of time, e.g. from about 5 to about 120 seconds, and left to incubate for a desired amount of time, e.g. at least from about 30 to about 120 seconds. At 312, the mix of blood and reagent is then applied to the aperture formed in the top layer of the test strip, as described above. At 314, wash buffer from the wash buffer vials of the kit is then applied to the aperture.

At 316, the reacted strip is then inserted into the optical sensing device to obtain a sample signal, such as a blue LED light reflectance and a red LED light reflectance. The application running on the mobile device uses the signals received from the optical sensing device to compute and display the HbA1c reading. In embodiments, the percentage of reflectance (% R) was obtained by dividing the sample signal with the blank signal. Percentage of reflectance obtained from red LED light represents HbA1c signals, while percentage of reflectance obtained from blue LED light represents total Hb signals. The measured reflectance values R were converted to a linearizing function K/S by the formula K/S=(1−R)(1−R)/2R. More information regarding the computations can be found, for example, in Džimbeg-malčić, V., Barbarić-mikočević, Ž. & Itrić, K. KUBELKA-MUNK THEORY IN DESCRIBING OPTICAL PROPERTIES OF PAPER (I); 1, 117-124 (2011) (Kubelka Munk theory), and Frantzen, F. et al. Glycohemoglobin filter assay for doctors' offices based on boronic acid affinity principle. Clin. Chem. 43, 2390-2396 (1997) (K/S computation for glycated hemoglobin), both of which are incorporated by reference in their entireties.

Optionally, graphic and/or text instructions illustrating this test procedure are shown to the user at each step on the mobile device's screen via the application. Upon completion, the test strip is disposed.

The example below was run using method 300 as one exemplary, non-limiting embodiment.

### Example 1

A blank test strip was first inserted into the device to obtain the background signal before the assay. The signal obtained from a blank test strip is defined as 100% reflectance. To start the assay, a 5 μl of blood sample was added to a tube containing 200 μl of lysis reagent. After sample was mixed, the tube was incubated for 2 min. 25 μl of the sample mix was applied from the tube onto the aperture of the test strip. Once the sample mix was absorbed by the test strip via the absorbent pad, 25 μl of wash buffer was applied to the aperture. Once the wash buffer was absorbed by the test strip, the test strip was inserted into the optical reader device, which in this example, included the portable POC Aina Device that is part of the Aina™ HbA1c Monitoring System available from Jana Med Tech Private Limited for Jana Care Inc. Red and Blue LED light sources contained within the device were automatically switched on by the device, and sample signal values were recorded in the phone.

The percentage of reflectance (% R) was obtained by dividing the sample signal with the blank signal. Percentage of reflectance obtained from red LED light represents HbA1c signals. Percentage of reflectance obtained from blue LED light represents total Hb signals. The measured reflectance values R were converted to a linearizing function K/S by the formula K/S=(1−R)(1−R)/2R.

TABLE 1
Red and blue LED reflectance values of blank and reacted test strips

| Bio-Rad Reference Reading | |
|---|---|
| Reflectance | Reflectance |
| Vaue of | Value of |
| Blank Strip | Reacted Strip |

| HbA1c % | Red | Blue | Red | Blue |
|---|---|---|---|---|
| 4.8 | 2612 | 4242 | 725 | 576 |
| 4.8 | 2623 | 4231 | 714 | 590 |
| 6.6 | 2607 | 4260 | 588 | 601 |
| 6.6 | 2602 | 4256 | 597 | 617 |
| 8.3 | 2609 | 4213 | 546 | 696 |
| 8.3 | 2622 | 4275 | 554 | 734 |
| 9.9 | 2570 | 4217 | 421 | 627 |
| 9.9 | 2565 | 4197 | 414 | 631 |
| 13 | 2604 | 4268 | 294 | 545 |
| 13 | 2627 | 4307 | 352 | 641 |

TABLE 2
Calculation of % HbA1c Readings

| % Reflectance | | K/S Value | | K/S Ratio | |
|---|---|---|---|---|---|
| Red | Blue | Red | Blue | Red/Blue | HbA1c % |
| 0.277565 | 0.135785 | 0.940162 | 2.750184 | 0.341854 | 4.71 |
| 0.272207 | 0.139447 | 0.972938 | 2.655317 | 0.366411 | 4.95 |
| 0.225547 | 0.141080 | 1.329610 | 2.614633 | 0.508526 | 6.34 |
| 0.229439 | 0.144972 | 1.293949 | 2.521432 | 0.513180 | 6.39 |
| 0.209276 | 0.165203 | 1.493832 | 2.109182 | 0.708252 | 8.29 |
| 0.211289 | 0.171696 | 1.472071 | 1.997973 | 0.736782 | 8.57 |
| 0.163813 | 0.148684 | 2.134163 | 2.437181 | 0.875669 | 9.91 |
| 0.161404 | 0.150345 | 2.178528 | 2.400846 | 0.907400 | 10.22 |
| 0.112903 | 0.127694 | 3.485023 | 2.979444 | 1.169689 | 12.73 |
| 0.133993 | 0.148827 | 2.798531 | 2.434008 | 1.149762 | 12.54 |

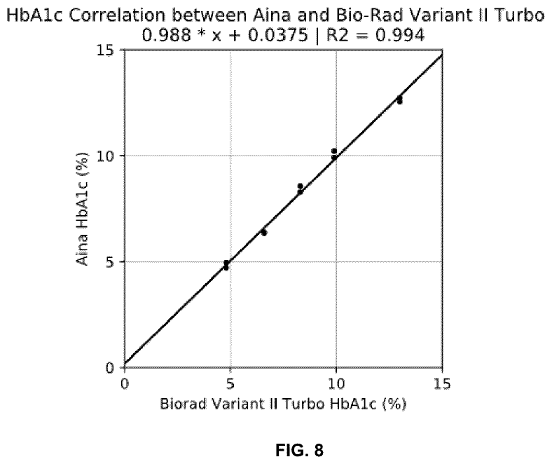

The % HbA1c results calculated from testing the blood samples on the Aina™ HbA1c device and also on the Bio-Rad Variant II Turbo™ instrument were subjected to linear regression analysis, as shown in FIG. 8, in which the linear regression was calculated to be R²=0.994, indicating a nearly linear relationship.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A test strip assembly for testing a blood sample in which red blood cells containing both glycated and non-glycated hemoglobin from the blood sample have been precipitated out from the blood sample using a reagent, the test strip assembly comprising:
a first film layer, the film layer including structure defining an aperture;
a porous membrane coupled to the first film layer and in fluid communication with the aperture, wherein the porous membrane is configured to retain the precipitated glycated and non-glycated hemoglobin particles and red blood cells from the blood sample, while allowing the remaining blood sample to pass through the membrane; and
a second film layer coupled to the porous membrane such that the porous membrane is positioned between the first substrate and the second film layer,
wherein the test strip assembly is configured to be positioned within an optical sensing device to determine a percentage of glycated hemoglobin of total hemoglobin in the blood sample.

2. The assembly of claim 1, further comprising:
an absorbent layer positioned between the porous membrane and the second film layer to aid in moving the blood sample from the aperture to and through the porous membrane.

3. The assembly of claim 1, further comprising:
a first bonding layer positioned between the first film layer and the porous membrane to bond the porous membrane to an inner surface of the first film layer such that the porous membrane is positioned below the aperture.

4. The assembly of claim 3, further comprising:
a second adhesive layer positioned between the second film layer and the absorbent layer to bond the porous membrane to an inner surface of the second film layer.

5. The assembly of claim 1, wherein the porous membrane is formed of a material selected from the group consisting of nitrocellulose, cellulose acetate, polyethylene, polyester, polyether sulfone, and combinations thereof.

6. The assembly of claim 1, wherein an axial length of the porous membrane is half or less of an axial length of the first film layer.

7. The assembly of claim 1, wherein a sidewall of the aperture is tapered, convex, or concave.

8. A composite sheet or roll comprising a plurality of test strip assemblies of claim 1.

9. A kit for monitoring diabetes using a blood sample, the kit comprising:
a plurality of test strip assemblies according to claim 1;
a plurality of vials containing the reagent configured to lyse red blood cells of the blood sample and precipitate glycated and non-glycated hemoglobin particles from the blood sample, the reagent containing a dye configured to conjugate only with the glycated hemoglobin particles; and
instructions for determining an amount of glycated hemoglobin from the blood sample, the instructions including:
collecting the blood sample from a patient;
combining the blood sample with the reagent to form a mixed blood sample;
applying a precipitated portion of the mixed blood sample to the aperture of the test strip assembly;
inserting the test strip assembly into an optical sensing device; and
operating the optical sensing device to determine the level of glycated hemoglobin in the sample.

10. The kit of claim 9, further comprising:
a plurality of washing solution, and
wherein the instructions further include, after applying the portion of mixed blood sample to the test strip assembly, applying a portion the washing solution to the aperture of the test strip assembly before inserting the test strip assembly into the optical sensing device.

11. The kit of claim 9, wherein during operation, the optical sensing device is configured to illuminate the test strip assembly with at least two different wavelengths to measure a reflected color at each of the at least two different wavelengths to measure glycated hemoglobin, and total hemoglobin.

12. The kit of claim 11, wherein the first wavelength is in a range of from about 600 nm to about 640 nm, and wherein the second wavelength is in a range of from about 450 nm to about 490 nm.

13. The kit of claim 9, wherein the instructions further include:
instructions for downloading or running an application on a user's mobile device, the application being configured to pair the optical sensing device and the mobile device such that the user can obtain data and analysis from the optical sensing device on the mobile device.

14. The test strip assembly of claim 1, wherein the first film layer is formed of a material that has a flexural modulus ranging from 100,000 psi to 600,000 and a tensile strength ranging from 3,000 psi to 15,000 psi.

15. The test strip assembly of claim 14, wherein the material is selected from the group consisting of acetal copolymer, acrylic, nylon, polyester, polypropylene, polyphenylene sulfide, polytehteretherketone (PEEK), PVC, and combinations thereof.

16. The test strip assembly of claim 1, wherein the second film layer is selected form the group consisting of polyethylene, PVC, polypropylene, PET, PTFE, and combinations thereof.

17. The test strip assembly of claim 2, wherein the absorbent layer comprises a woven or non-woven material selected from the group consisting of nylon, fiberglass, cellulose, and combinations thereof.

18. The test strip assembly of claim 17, wherein the absorbent layer comprises one-direction woven fiber.

19. The test strip assembly of claim 1, wherein the first and/or second bonding layers comprises an acrylic polymer.

20. The test strip assembly of claim 1, wherein the porous membrane includes a whitening agent to induce opacity of the porous membrane.

21. A method of monitoring diabetic patients, the method comprising:
obtaining a blood sample from a patient;
combining the blood sample with a reagent configured to lyse red blood cells in the blood sample and to precipitate hemoglobin and glycated hemoglobin from the blood sample;
applying a portion of the reacted blood sample to the aperture of a test strip assembly of claim 1; and
inserting the test strip assembly into an optical sensing device operably coupled to an optical reader device to obtain a value of glycated hemoglobin from the blood sample.

22. The method of claim 21, wherein the reagent contains a dye configured to conjugate with glycated hemoglobin only.

23. The method of claim 22, wherein the dye comprises a blue dye containing boronic acid.

24. The method of claim 21, wherein the optical sensing device comprises a first light source for illuminating the test strip at a first wavelength to measure a first color reflectance, and a second light source for illuminating the test strip at a second wavelength to measure a second color reflectance, wherein the first color reflectance indicates a level of glycated hemoglobin, and the second color reflectance indicates a level of total hemoglobin.

25. The method of claim 21, the method further comprising:
providing an analyzer device comprising a mobile device;
opening an application installed on the mobile device;
pairing the mobile device and the optical sensing device such that information can be communicated between devices; and
reading data and/or analysis generated by the optical sensing device on the mobile device.
